(19) United States
(12) Patent Application Publication
(10) Pub. No.: US 2019/0321345 A1
(43) Pub. Date: Oct. 24, 2019

(54) GLUT4 Selective Inhibitors for Cancer Therapy

(71) Applicants: Emory University, Atlanta, GA (US); Northwestern University, Evanston, IL (US); Washington University, St. Louis, MO (US)

(72) Inventors: Malathy Shanmugam, Atlanta, GA (US); Gary Schiltz, Naperville, IL (US); Rama Mishra, Chicago, IL (US); Paul Hruz, Manchester, MO (US)

(21) Appl. No.: 16/475,062

(22) PCT Filed: Dec. 28, 2017
(86) PCT No.: PCT/US2017/068648

§ 371 Date: Jun. 28, 2019

Related U.S. Application Data
(60) Provisional application No. 62/439,801, filed on Dec. 28, 2016.

(51) Int. Cl. A61K 31/444; C07D 213/16; C07D 405/12; C07D 403/12; C07D 403/14; A61K 31/438; A61K 31/4433; A61K 31/4409; A61P 35/00; A61K 31/496; A61K 31/573; A61K 31/197

(52) U.S. Cl. CPC A61K 31/444; C07D 213/16; C07D 405/12; C07D 403/12; C07D 403/14; A61K 31/197; A61K 31/4433; A61K 31/4409; A61P 35/00; A61K 31/496; A61K 31/573; A61K 31/438

(57) ABSTRACT

This disclosure relates to GLUT 4 inhibitors and uses as chemotherapy agents. In certain embodiments, this disclosure relates to methods of treating or preventing cancer comprising administering an effective amount of a GLUT 4 inhibitor disclosed herein to a subject in need thereof. In certain embodiments, the GLUT 4 inhibitor has Formula (I), prodrugs, derivatives, or salts thereof wherein the substituents are reported herein. In certain embodiments, the GLUT 4 inhibitor is N-(3-(4-fluorophenethoxy)benzyl)-2-(4-methoxyphenyl)-N-(pyridin-4-ylmethyl)acetamide or salts thereof.

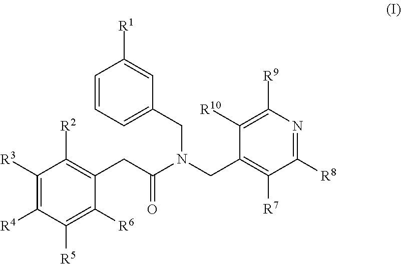

### CROSS-REFERENCE TO RELATED APPLICATIONS

This application claims the benefit of U.S. Provisional Application No. 62/439,801 filed Dec. 28, 2016. The entirety of this application is hereby incorporated by reference for all purposes.

### STATEMENT REGARDING FEDERALLY FUNDED RESEARCH

This invention was made with government support under P30 CA060553 awarded by the National Institutes of Health. The government has certain rights in the invention.

### BACKGROUND

Tumor cells, including those of the largely fatal plasma cell malignancy, multiple myeloma (MM), exhibit elevated glucose uptake. The fundamental reliance of tumor cells on increased glucose catabolism for survival, proliferation and chemoresistance is well established. MM cells surprisingly exhibit increased constitutive expression of GLUT4 on the plasma membrane, co-opting use of this transporter (among the 14 GLUTs) and not GLUT1 for survival and proliferation. GLUT4 inhibition abrogates cell proliferation and chemoresistance in vitro in MM, chronic lymphocytic leukemia (CLL), solid tumor lines. Roles for GLUT4 have also been suggested in human gastrointestinal tumors and in breast cancers.

Mishra et al. report silico modeling-based identification of glucose transporter 4 (GLUT4)-selective inhibitors for cancer therapy J. Biol. Chem., 290 (23) (2015), pp. 14441-14453.

Aurora Fine Chemicals LLC report a commercial source of N-[[3-[2-(4-fluorophenyl)ethoxy]phenyl]methyl]-N-(4-pyridinylmethyl)-1,3-benzodioxole-5-acetamide, CAS registry number 1060458-38-9.

References cited herein are not an admission of prior art.

### SUMMARY

This disclosure relates to GLUT 4 inhibitors and uses as chemotherapy agents. In certain embodiments, this disclosure relates to methods of treating or preventing cancer comprising administering an effective amount of a GLUT 4 inhibitor disclosed herein to a subject in need thereof. In certain embodiments, the GLUT 4 inhibitor has Formula I:

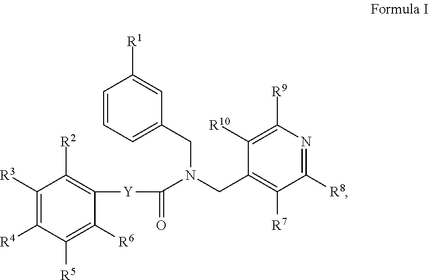

prodrugs, derivatives, or salts thereof wherein the substituents are reported herein. In certain embodiments, the GLUT 4 inhibitor is N-(3-(4-fluorophenethoxy)benzyl)-2-(4-methoxyphenyl)-N-(pyridin-4-ylmethyl)acetamide or salts thereof.

In certain embodiments, this disclosure relates to pharmaceutical compositions comprising a compound disclosed herein, or derivative thereof, and a pharmaceutically acceptable excipient. In certain embodiments, the pharmaceutical composition is in the form of a pill, tablet, capsule, cream, or saline-buffered solution. In certain embodiments, the pharmaceutically acceptable excipient is a saccharide or polysaccharide.

In certain embodiments, this disclosure relates to method of treating or preventing cancer comprising administering an effective amount of a compound disclosed herein, or derivative thereof, to a subject in need thereof in combination with a second chemotherapeutic agent. In certain embodiments, the second chemotherapeutic agent is venetoclax, melphalan, dexamethasone, or combinations thereof.

### BRIEF DESCRIPTION OF THE DRAWINGS

FIG. 1 shows GLUT4 antagonists.

FIG. 2A shows data on a screen for identifying GLUT4-selective inhibitors. KMS11 isogenic myeloma cell lines express GFP or GLUT1.

FIG. 2B illustrates JJN3 cells expressing non-targeting pLKO.1 or GLUT4 directed shRNA are treated with test compounds for 72 hours following which viability is assessed using the Cell Titre Glo assay.

FIG. 3A shows data on Compound 20 inhibiting glucose uptake in JJN3 and reducing cell proliferation by targeting GLUT4. JJN3 cells were transduced with non-targeting or GLUT4-directed shRNA. Efficiency of knockdown was determined by q-RTPCR analysis of GLUT4 normalized to GAPDH expression.

FIG. 3B shows data for evaluation of 2-[3H]deoxyglucose (2-DOG) uptake: C shRNA and GLUT4 shRNA expressing cells were pre-treated with Compound 20 (20 μM) for 6 min followed by measurement of 2-DOG uptake for 6 min at 37 degrees C.

FIG. 3C shows data on cells evaluated for viability/proliferation after 72 hours of Compound 20 (15 μM) treatment using trypan blue and an automated cell counter.

FIG. 4 shows data indicating that compound 20 exhibits inhibition of 2-deoxyglucose (2-DOG) transport through GLUT4. Following a 5-minute exposure to Compound 20, HEK cells over-expressing individual human GLUTs were assayed for 2-DOG uptake for 4 minutes at 37° C. Non-specific uptake was measured in non-transfected HEK293 cells containing the shRNA GLUT1 knockdown and was subtracted from the experimental values.

FIG. 5 shows data indicating compound 20 selectively binding to GLUT4 vs. GLUT1. DMSO (vehicle), compound 26 (20 μM) and compound 20 (20 μM) were added to 200 μg LDM prepared from Myc-tagged GLUT1- or GLUT4-overexpressing cells for 10 min at room temperature. Samples were then incubated with biotinylated ATB-BMPA (50 μM final concentration) followed by UV irradiation. Biotinylated proteins, isolated from detergent solubilized LDM using a high-capacity streptavidin agarose resin, were analyzed by immunoblot analysis using GLUT4 or GLUT1 antibodies. GLUT proteins were quantified using an Odyssey Infrared Imaging System. Compound 26 denoted with an asterisk, a reported GLUT4 antagonists (Mishra et al., J. Biol. Chem., 2015) was used as a positive control.

FIG. 6A shows data immunoblot analysis of cellular lysates when JJN3 and L363 cells were treated with/with-out compound 20 (15 μM for 18 hours were evaluated for expression of MCL-1 and actin (as a loading control)).

FIG. 6B shows data on L363 cells treated with 10 μM compound 20 with/without indicated concentrations (0.025 μM-3 μM) of ABT-199 for 72 hours were evaluated for cell death. Compound 20 decreasing MCL-1 expression and sensitizing MM cell lines to Venetoclax (ABT-199),

FIG. 6C shows data on JJN3 cells.

FIG. 6D shows data on MM patient sample bone marrow aspirate buffy coat cells were similarly treated with 10 μM compound 20 with or without 0.5 μM ABT-199 for 48 hours. CD38-phyocerythrin and CD45-allophycocyanin-Cy7 positive myeloma cells were evaluated for cell death.

FIG. 6E shows data on MM.1S cells that were treated with indicated concentrations of dexamethasone (Dex) (0.00195 μM-1 μM) and/or 10 μM compound 20 for 72 followed by assessment of cell death.

FIG. 6F shows data on MM patient samples treated with 10 μM compound 20 and 0.5 μM Dex for 48 hr and evaluation of cell death.

FIG. 6G shows data on effect of compound 20 administered in combination with ABT-199 or Dex on normal cellular populations within MM patient bone marrow aspirate buffy coat cells used in (6F). Data from patient #6 is representative of patient samples evaluated.

FIG. 6H shows data on MM.1S cells treated with 10 μM compound 20 or the indicated concentrations (0.01 μM-4 μM) of melphalan (Mel) or their combination for 72 hours were evaluated for cell death. Cell death was evaluated by AnnexinV/DAPI flow cytometric staining.

FIG. 7 shows data indicating suppression of GLUT4 inhibits follower lung cancer cell invasion using compound 20.

FIG. 8 shows data indicating suppression of GLUT4 inhibits FAK activation using a 071917 scratch assay using compound 20.

FIG. 9 shows data treatment of compound 26 inhibits H1299 cell invasion (human lung cells derived from metastatic lymph node).

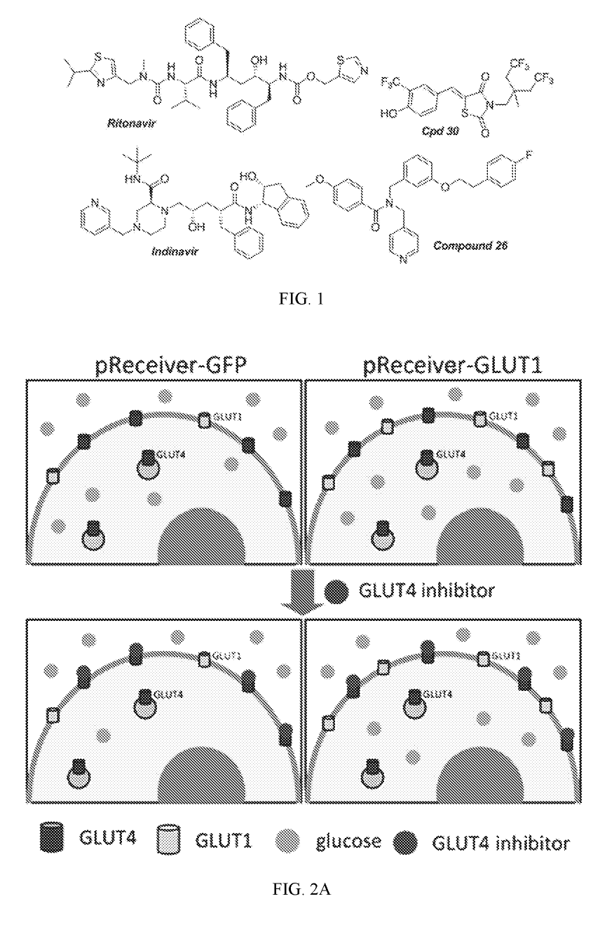

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

In this specification and in the claims that follow, reference will be made to a number of terms that shall be defined to have the following meanings unless a contrary intention is apparent.

As used herein, the term “combination with” when used to describe administration with an additional treatment means that the agent may be administered prior to, together with, or after the additional treatment, or a combination thereof, however provides a therapeutic amount of both agents, e.g., both at sufficient concentrations in the blood at the same time.

As used herein, “subject” refers to any animal, typically a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” can be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample particle compared to a control without the particle. It can also be identified by a change in relevant biomarker or gene expression profile, such as PSA for prostate cancer, HER2 for breast cancer, or others.

As used herein, “alkyl” means a noncyclic straight chain or branched, unsaturated or saturated hydrocarbon such as those containing from 1 to 10 carbon atoms. A “higher alkyl” refers to unsaturated or saturated hydrocarbon having 6 or more carbon atoms. A “C₈-C₁₈” refers to an alkyl containing 8 to 18 carbon atoms. Likewise a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or “heterocarbocyclyl” groups are carbocycles which contain from 1 to 4 heteroatoms independently selected from nitrogen, oxygen and sulphur which may be saturated or unsaturated (but not aromatic), monocyclic or polycyclic, and wherein the nitrogen and sulphur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, propoxy, n-butoxy, s-butoxy, t-butoxy.

“Alkoxyalkyl” refers an alkyl group as defined above with the indicated number of carbon atoms attached through an alkyl bridge (i.e., —CH₂—O—CH₂CH₃).

“Alkylamino” refers an alkyl group as defined above with the indicated number of carbon atoms attached through an amino bridge. An example of an alkylamino is methylamino, (i.e., —NH—CH₃).

“Alkylthio” refers to an alkyl group as defined above with the indicated number of carbon atoms attached through a sulfur bridge. An example of an alkylthio is methylthio, (i.e., —S—CH₃).

“Alkanoyl” refers to an alkyl as defined above with the indicated number of carbon atoms attached through a carbonyl bride (i.e., —(C═O)alkyl).

The terms “cycloalkyl” and “cycloalkenyl” refer to mono-, bi-, or tri homocyclic ring groups of 3 to 15 carbon atoms which are, respectively, fully saturated and partially unsaturated.

The terms “halogen” or “Hal” refer to fluorine, chlorine, bromine, and iodine.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, adding a hydroxyl group, replacing an oxygen atom with a sulfur atom, or replacing an amino group with a hydroxyl group, oxidizing a hydroxyl group to a carbonyl group, reducing a carbonyl group to a hydroxyl group, and reducing a carbon-to-carbon double bond to an alkyl group or oxidizing a carbon-to-carbon single bond to a double bond. A derivative optional has one or more, the same or different, substitutions. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

### Development of GLUT4 Selective Inhibitors for Cancer Therapy

Although it is not intended that embodiments of this disclosure be limited by any particular mechanism, it is believed that treatment and chemosensitization of multiple myeloma and other cancers is associated with a mechanism of pharmacological inhibition of GLUT4 and thereby produce agents that specifically target cancer cells that rely on glucose transport via GLUT4.

Studies demonstrate glucose uptake into tumor cells to be rate-limiting step in glucose metabolism. GLUT1 is elevated in a number of cancers; however, being widely expressed and a major glucose transporter in erythrocytes and the blood brain barrier due to expression in neurons and endothelial cells, it undermines the utility of targeting GLUT1 for cancer therapy. GLUT4 is expressed in muscle (skeletal and heart) and adipose tissue and plays a central role in whole body glucose homeostasis by facilitating insulin- and exercise-stimulated glucose transport. GLUT4 is basally retained within the cell with less than 1% localized to the plasma membrane of skeletal myocytes, adipocytes and cardiac myocytes. GLUT4 facilitates glucose transport into the cell only when it is translocated from intracellular compartments to the plasma membrane. In fact, it is the inability to recruit sufficient GLUT4 to the plasma membrane in muscle and fat cells that accounts for hyperglycemia in people with diabetes mellitus. The complex trafficking of GLUT4 to the plasma membrane is facilitated by the PI3K/AKT6 and AMPK pathways. It is therefore not surprising that tumor cells dependent on sustained PI3K/AKT or AMPK activation could exhibit increased levels of constitutive GLUT4 expression on the plasma membrane to support elevated glucose uptake even in the absence of insulin stimulation.

Studies demonstrate the presence of elevated GLUT4 in the plasma membrane of multiple myeloma cells and responsiveness of a number of solid and liquid tumor cell lines to GLUT4 inhibition. Our current studies demonstrate that targeting GLUT4 in addition to being a cytotstatic and/or cytotoxic can be used to sensitize cancer cells to existing chemotherapy (dexamethasone and melphalan) and BH3 mimetics, affording potential strategies for chemosensitization and synthetic lethality, respectively.

The normal physiologic role of GLUT4 in whole-body glucose homeostasis and consequences of genetic and pharmacologic ablation is well-established both in rodent models and humans. Since GLUT4 is retained within the cell during unstimulated basal conditions, basal glucose levels are unchanged in whole body and muscle-specific GLUT4-null mice. Whole body and muscle-specific GLUT4 null mice exhibit a reduction in fat accumulation, hyperinsulinemia and increased gluconeogenesis in the liver with time, associated with prolonged hyperglycemia and hyperinsulinemia. Significant growth retardation and reduced survival is detected after six months in a whole body GLUT4-null mouse model.

Chemoresistance in MM, leukemia, and solid tumors is largely due to tumor cell evasion of apoptosis that in turn is primarily due to the inability to release sufficient pro-apoptotic BCL-2 proteins above a threshold level required to elicit apoptosis. Since BCL-2 family members are central to the development of chemotherapeutic resistance, the ability to selectively perturb glucose metabolism in cancer cells provides a method to not only suppress resistance-promoting MCL-1 expression; but also elicit synthetic lethality to BH3 mimetics like ABT-199.

A homology model of GLUT4 was used to carry out an in silico screen to identify several GLUT4 antagonists. A diverse substituted aromatic and non-aromatic groups was selected that possessed varying hydrogen-bond acceptor capacity and electron donating/withdrawing ability. This route allowed exploring the SAR for this series to improve their biological properties. Several analogs of the aryl amide were prepared. Interestingly, inclusion of a methylene spacer in the amide portion (e.g. compound 20) produced increased selectivity in our phenotypic screening assay while maintaining similar potency as other analogs. This compound was then used in other assays to show the effects of GLUT4 inhibition on a variety of MM-related properties.

Targeting GLUT4 in MM leads to apoptosis in a subset of MM cells associated with suppression of the resistance promoting BCL-2 family member MCL-1. MM cells resistant to the cytotoxic effects of GLUT4 inhibition were found to induce chemosensitizing alterations in BCL-2 proteins, supporting the use of GLUT4 inhibitors as both therapeutic agents and chemosensitizers.

MM cells exhibit increased constitutive expression of GLUT4 on the plasma membrane, co-opting use of this transporter (among the 14 GLUTs) and not GLUT1 for survival and proliferation. GLUT4 inhibition abrogates cell proliferation and chemo-resistance in vitro in MM, chronic lymphocytic leukemia (CLL), solid tumor lines and in vivo in a xenograft model of MM. Roles for GLUT4 have also been suggested in human gastrointestinal tumors that exhibit enhanced PM localization of GLUT4 and weak expression of GLUT112 and in breast cancers. The observations suggest GLUT4 serves a unique role in both solid and liquid cancers.

### GLUT4 Selective Inhibitors

In certain embodiments, the GLUT4 inhibitor of the instant disclosure has Formula I:

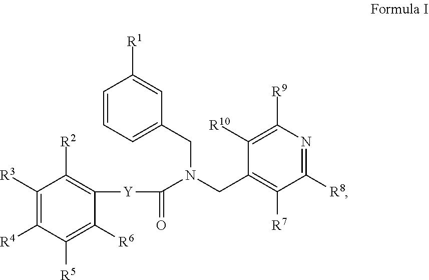

or salts thereof, wherein

Y is —CH₂—, or a direct bond from the carbonyl to the phenyl ring;

R¹, R², R³, R⁴, R⁵, R⁶, R⁷, R⁸, R⁹, and R¹⁰ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R¹, R², R³, R⁴, R⁵, R⁶, R⁷, R⁸, R⁹, and R¹⁰ are optionally substituted with one or more, the same or different, R¹¹;

R¹¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹²; and

R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R¹ is alkoxy substituted with aryl, and wherein the aryl group is optionally substituted with a halogen.

In certain embodiments, Formula I is Formula IA

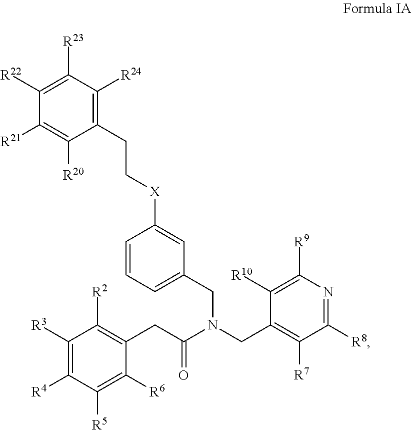

prodrugs, derivatives, or salts thereof, wherein

X is O, S, or NH;

R², R³, R⁴, R⁵, R⁶, R⁷, R⁸, R⁹, and R¹⁰ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R², R³, R⁴, R⁵, R⁶, R⁷, R⁸, R⁹, and R¹⁰ are optionally substituted with one or more, the same or different, R¹¹;

R¹¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹²;

R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R²⁰, R²¹, R²², R²³, and R²⁴ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R²⁰, R²¹, R²², R²³, and R²⁴ are optionally substituted with one or more, the same or different, R³¹;

R³¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³¹ is optionally substituted with one or more, the same or different, R³²; and

R³² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the compound of formula I is N-(3-(4-fluorophenethoxy) benzyl)-2-(4-methoxyphenyl)-N-(pyridin-4-ylmethyl)acetamide (compound 20) or salts thereof. In certain embodiments, this disclosure relates to a compound disclosed herein substituted with one or more substituents.

In certain embodiments, R¹ is alkoxy substituted with a hetercyclyl, and wherein the heterocyclyl group is optionally substituted with R¹¹.

In certain embodiments, Formula I is Formula 1B

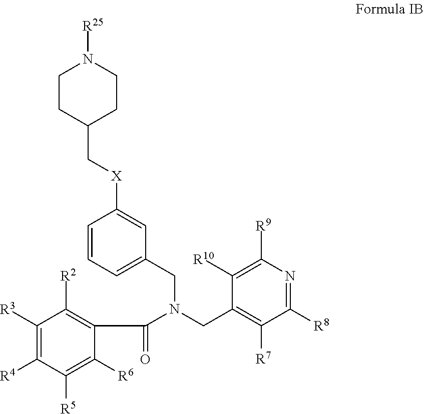

prodrugs, derivatives, or salts thereof, wherein

X is O, S, or NH;

R², R³, R⁴, R⁵, R⁶, R⁷, R⁸, R⁹, and R¹⁰ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R², R³, R⁴, R⁵, R⁶, R⁷, R⁸, R⁹, and R¹⁰ are optionally substituted with one or more, the same or different, R¹¹;

R¹¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein is optionally substituted with one or more, the same or different, R¹²;

R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R²⁵ is hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R²⁵ is optionally substituted with one or more, the same or different, R³¹;

R³¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³¹ is optionally substituted with one or more, the same or different, R³²; and

R³² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

### Methods of Use

This disclosure relates to methods of pharmacological inhibition of GLUT4 as a strategy for the treatment and chemosensitization of multiple myeloma and other cancers. In certain embodiments, the disclosure relates to methods of treating or preventing cancer comprising administering a pharmaceutical composition comprising GLUT4 inhibitors disclosed herein to a subject diagnosed with, exhibiting symptoms of, or at risk of cancer. In certain embodiments, the cancer is selected from the group consisting of leukemia, melanoma, cervical, ovarian, colon, breast, gastric, lung, skin, ovarian, pancreatic, prostate, head, neck, and renal cancer.

In certain embodiments, the pharmaceutical composition is administered in combination with a second chemotherapeutic agent such as, but not limited to, gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin bortezomib anegrilide, tamoxifen, toremifene, raloxifene, droloxifene, iodoxyfene fulvestrant, bicalutamide, flutamide, nilutamide, cyproterone, goserelin, leuprorelin, buserelin, megestrol anastrozole, letrozole, vorazole, exemestane, finasteride, marimastat, trastuzumab, cetuximab, dasatinib, imatinib, bevacizumab, combretastatin, thalidomide, and/or lenalidomide or combinations thereof.

In certain embodiments, the disclosure relates to methods of treating or preventing multiply myeloma comprising administering a pharmaceutical composition comprising GLUT4 inhibitors disclosed herein to a subject in need thereof in combination with melphalan, vincristine, cyclophosphamide, etoposide, doxorubicin, liposomal doxorubicin, bendamustine, or combinations thereof.

In certain embodiments, the disclosure relates to methods of treating or preventing multiply myeloma comprising administering a pharmaceutical composition comprising GLUT4 inhibitors disclosed herein to a subject in need thereof in combination with a bisphosphonate such as pamidronate or zoledronic acid.

In certain embodiments, the disclosure relates to therapeutic methods disclosed herein wherein the pharmaceutical compositions are administered before, after or during radiotherapy.

In certain embodiments, the disclosure relates to uses of compounds disclosed herein in the production of a medicament for the treatment or prevention of cancer.

### Formulations

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When a compound contains a hydrogen-donating heteroatom (e.g. NH), salts are contemplated to covers isomers formed by transfer of said hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, esters, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. In certain embodiments, the compound is administered by inhalation through the lungs.

The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778, 6,369,086, 6,369,087 and U.S. Pat. No. 6,372,733 and the further references mentioned above, as well as to the standard handbooks, such as the latest edition of Remington's Pharmaceutical Sciences.

Depending upon the manner of introduction, the compounds described herein may be formulated in a variety of ways. Formulations containing one or more compounds can be prepared in various pharmaceutical forms, such as granules, tablets, capsules, suppositories, powders, controlled release formulations, suspensions, emulsions, creams, gels, ointments, salves, lotions, or aerosols and the like. Preferably, these formulations are employed in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized. In another embodiment, the formulation is administered topically. Suitable topical formulations include, but are not limited to, lotions, ointments, creams, and gels. In a preferred embodiment, the topical formulation is a gel. In another embodiment, the formulation is administered intranasally.

In certain embodiments, the pharmaceutical composition comprises a compound disclosed herein and a propellant. In certain embodiments, an aerosolizing propellant is compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane or combinations thereof.

In certain embodiments, the disclosure contemplates a pressurized or unpressurized container comprising a compound herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicon dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-beta-iminodipropionate, myristoamphoacetate, lauryl betaine, and lauryl sulfobetaine.

If desired, the tablets, beads, granules, or particles may also contain minor amount of nontoxic auxiliary substances such as wetting or emulsifying agents, dyes, pH buffering agents, or preservatives.

### Experimental

### Cell Proliferation Assays and Viability Assays

IC₅₀ studies were performed using the CellTiter-Glo assay (Promega, Madison, Wis.). 20,000 cells in 100 ul complete RPMI 1640 medium were plated per well in 96-well plates, with a concentration range of individual compounds. After 72 h incubation, 100 uL Glo reagent were added to each well and luminescence was measured using a Biotek Synergy 4 multimode plate reader. Cell number was assessed by trypan blue standing and an automatic cell counter (Biorad). For cell viability assays 0.125×10⁶ cells/mL were treated with the indicated concentration of drug, washed with 1×PBS and evaluated for viability by AnnexinV/DAPI staining and flow cytometry. Data analysis was per-formed with the FCS express version 3 (De Novo software, Los Angeles).

### 2-Deoxyglucose Uptake Measurements

Tissue culture plates were pretreated with 25 μg/ml polyethyleneimine (Fluka, catalogue number P3143) in 150 mM NaCl for 20min to let cells adhere. JNN3 cells were subsequently plated and grown to 40-60% confluency in complete RPMI 1640 medium. HEK293 GLUT overexpressing cells were plated at 400,000 cells/ml overnight. Cells were then washed with glucose-free HEPES buffer twice and starved for 30 min. The uptake of 2-[³H] deoxy-D-glucose (2-DOG) (50 μM) was measured in glucose-free HEPES buffer for 4-6 min at 37° C. Compound 20 (20 μM) was added 5-6 min prior to the addition of glucose depending on the specific assay. For the HEK293 GLUT overexpressing cells, non-specific uptake was measured in non-transfected HEK293 cells containing the shRNA GLUT1 knockdown and was subtracted from the experimental values. Data are plotted as percent uptake relative to unexposed HEK293 cultures. Data were normalized to fit by nonlinear regression analysis using GraphPad Prism software.

### Isolation and Photolabeling of Low Density Microsomes

LDMs were isolated from Myc-tagged GLUT1 or GLUT4 cells. Inhibitors were added to LDMs (200-400 g) for 10 min at room temperature. Samples were then incubated for 10 min at room temperature with biotinylated ATB-BMPA (50 M final concentration) ATB-BMPA (2.5 M final concentration) and then placed on ice prior to UV irradiation. Reactions were transferred to a 24-well low protein retention culture dish (Costar, Corning, N.Y.) and then irradiated at room temperature 5 cm from a Green Spot UV lamp for 1 min (30 s of light followed by 30 s of darkness followed by 30 s of light).

### Synthesis of Tertiary Amide Compounds

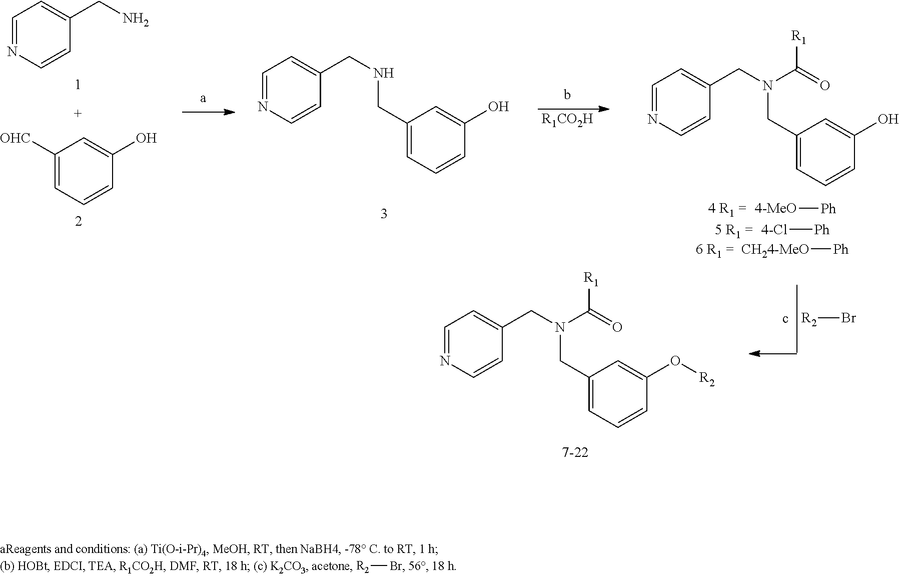

### Preparation of Intermediates and Final Compounds

### 3-(((Pyridin-4-ylmethyl)amino)methyl)phenol (3)

To a solution of pyridin-4-ylmethanamine (2.49 mL, 24.57 mmol) and 3-hydroxy benzaldehyde (3.0 g, 24.57 mmol) in MeOH (60 mL) was added tetraisopropoxytitanium (9.36 mL, 31.9 mmol). The reaction was stirred for overnight (16 h) and then cooled to −78° C. Sodium borohydride (0.929 g, 24.57 mmol) was then added and reaction mixture stirred for another 1 h. The reaction was then quenched with water and extracted with ethyl acetate. The organic layer was evaporated and dried in vacuum. The reaction mixture was purified by flash chromatography (0-20% MeOH/DCM) to give the title compound (4.45 g, 85%). Rf in 10% MeOH/DCM=0.5; MS (ESI): mass calcd. for C₁₃H₁₄N₂O, 214.11; m/z found, 215.18 [M+H]+; ¹H NMR (500 MHz, CDCl₃) δ ppm 3.80 (s, 2 H), 3.87 (s, 2 H), 6.79-6.82 (m, 1 H), 6.86 (d, J=6.71 Hz, 1 H), 6.92-6.96 (m, 1 H), 7.23 (t, J=7.78 Hz, 1 H), 7.35-7.38 (m, 2 H), 8.56-8.59 (m, 2 H).

### N-(3-hydroxybenzyl)-4-methoxy-N-(pyridin-4-ylmethyl)benzamide (4)

To a mixture of 4-methoxybenzoic acid (1.015 g, 6.67 mmol), HOBT (1.022 g, 6.67 mmol), EDC (1.279 g, 6.67 mmol), and 3-(((pyridin-4-ylmethyl)amino)methyl)phenol (1.3 g, 6.07 mmol) in DMF (25 mL) was added Et₃N (0.863 mL, 6.19 mmol). The reaction was stirred overnight (18 h). After the reaction, water was added and reaction was extracted with ethyl acetate. The organic layer was evaporated and dried in vacuum. The crude was purified using flash chromatography (0-20% MeOH/DCM) to give the title compound (0.9 g, 43%). Rf in 5% MeOH/DCM=0.45; MS (ESI): mass calcd. for C₂₁H₂₀N₂O₃, 348.15; m/z found, 349.41 [M+H]+; ¹H NMR (500 MHz, CDCl₃) δ 3.82 (s, 3 H), 4.37-4.78 (m, 4 H), 6.59-6.78 (m, 2 H), 6.80-6.92 (m, 3 H), 7.12-7.27 (m, 3 H), 7.35-7.57 (m, 2 H), 8.59 (d, J=4.27 Hz, 2 H).

### 4-Chloro-N-(3-hydroxybenzyl)-N-(pyridin-4-ylmethyl)benzamide (5)

3-(((pyridin-4-ylmethyl)amino)methyl)phenol (150 mg, 0.700 mmol) was dissolved in THF (2.0 ml). Then Et₃N (0.081 ml, 0.583 mmol) was added and reaction stirred for 5 min. 4-chlorobenzoyl chloride (0.075 ml, 0.583 mmol) was then added and reaction stirred overnight (12 h). After the reaction, water was added and the reaction mixture was extracted with ethyl acetate. The organic layer was evaporated and dried in vacuo. The crude was purified using flash chromatography (0-20% MeOH/DCM) to give the title compound (74 mg, 36%). MS (ESI): mass calcd. for C₂₀H₁₇ClN₂O₂, 352.10; m/z found, 353.36 [M+H]+; ¹H NMR (500 MHz, CDCl₃) δ 4.32-4.49 (m, 2 H), 4.61-4.75 (m, 2 H), 6.59-6.80 (m, 2 H), 6.83 (d, J=9.16 Hz, 1 H), 7.11-7.27 (m, 3 H), 7.33-7.49 (m, 4 H), 8.59 (br. s., 2 H).

### N-(3-hydroxybenzyl)-2-(4-methoxyphenyl)-N-(pyridin-4-ylmethyl)acetamide (6)

To a mixture of 2-(4-methoxyphenyl)acetic acid (128 mg, 0.770 mmol), HOBT (118 mg, 0.77 mmol), EDC (148 mg, 0.77 mmol), and 3-(((pyridin-4-ylmethyl)amino)methyl)phenol (150 mg, 0.70 mmol) in DMF (2 ml) was added Et₃N (0.117 ml, 0.84 mmol). The reaction was stirred overnight (18 h). Afterwards, water was added and the reaction was extracted with ethyl acetate. The organic layer obtained was evaporated and dried in vacuum. The crude residue was used as is in the next step.

A sample was purified by preparative HPLC to allow ¹H NMR characterization. The sample appeared as a 2:1 mixture of rotamers. ¹H NMR (500 MHz, CDCl₃) δ ppm 3.69 (s, 1 H), 3.75 (s, 1.5 H), 3.77 (s, 2 H), 3.79 (s, 3 H), 4.41 (s, 2 H), 4.44 (s, 1 H), 4.54 (s, 1 H), 4.57 (s, 2 H), 6.58-6.62 (m, 2 H), 6.65 (d, J=7.63 Hz, 0.5 H), 6.72 (s, 0.5 H), 6.77 (dd, J=8.24, 1.83 Hz, 0.5 H), 6.81 (d, J=8.85 Hz, 2 H), 6.84-6.88 (m, 2 H), 7.00 (d, J=5.80 Hz, 1H), 7.08 (d, J=5.80 Hz, 2 H), 7.11-7.15 (m, 1.5 H), 7.16-7.22 (m, 2 H), 8.46-8.49 (m, 2 H), 8.54 (d, J=6.10 Hz, 1 H).

### Representative Procedure for the Synthesis of Final Compounds

Acetone (0.5 mL) was added to phenol (4-6) (50 mg, 0.144 mmol). Then K₂CO₃ (39.7 mg, 0.287 mmol, 2eq.) and bromide (0.144 mmol, 1 eq.) were added and the reaction mixture was stirred at 56° C. overnight. Thereafter, the reaction mixture was filtered, centrifuged, and the supernatant was evaporated to dryness. The crude was purified using prep TLC or Biotage flash column chromatography to afford the desired final compound.

### 4-Methoxy-N-(3-(4-methylphenethoxy)benzyl)-N-(pyridin-4-ylmethyl)benzamide (7)

Yield=32%. ¹H NMR (500 MHz, CD₃OD) δ 2.29 (s, 3 H), 2.98 (t, J=6.87 Hz, 2 H), 3.31 (dt, J=3.28, 1.56 Hz, 2 H), 3.79 (s, 3 H) 4.10 (m, 2 H), 4.51-4.75 (m, 4 H), 6.58-6.67 (m, 1 H), 6.70-6.78 (m, 1 H), 6.82 (d, J=6.71 Hz, 1 H), 6.95 (d, J=7.93 Hz, 2 H), 7.07-7.11 (m, 2 H), 7.14-7.17 (m, 2 H), 7.22 (t, J=7.93 Hz, 1 H), 7.26-7.34 (m, 2 H), 7.46 (d, J=7.93 Hz, 2 H), 8.45 (d, J=6.10 Hz, 2 H); ¹³C NMR (126 MHz, CDCl₃) δ 21.03, 35.30, 55.33, 68.86, 113.63, 113.88, 113.94, 127.42, 128.86, 129.19, 129.95, 134.92, 136.09, 137.86, 146.21, 150.14, 159.36, 161.00, 172.25. HRMS (ESI): m/z calculated for C₃₀H₃₀N₂O₃+H⁺ [M+H⁺]: 467.2329. Found: 467.2337.

### 4-Methoxy-N-(pyridin-4-ylmethyl)-N-(3-(3-(trifluoromethyl)phenethoxy)benzyl)benzamide (8)

Yield=12%. ¹H NMR (500 MHz, CD₃OD) δ ppm 3.17 (t, J=6.41 Hz, 2 H), 3.83 (s, 3 H), 4.21 (br. s., 2 H), 4.57-4.78 (m, 4 H), 6.62-6.92 (m, 3 H), 6.94-7.04 (m, 2 H), 7.21-7.56 (m, 8 H), 7.60 (d, J=7.32 Hz, 1 H), 7.65 (s, 1 H), 8.51 (br. s., 1 H); ¹³C NMR (126 MHz, CD₃OD) δ 29.27, 34.94, 54.46, 67.89, 113.58, 113.65, 122.80, 122.83, 125.38, 125.42, 125.45, 128.76, 130.11, 130.37, 132.53, 140.06, 159.23, 161.38, 173.40. HRMS (ESI): m/z calculated for C₃₀H27F₃N₂O₃+H⁺ [M+H⁺]: 521.2047. Found: 521.2053.

### 4-Methoxy-N-(pyridin-4-ylmethyl)-N-(3-((tetrahydro-2H-pyran-4-yl)methoxy)benzyl)benzamide (9)

Yield=52%. ¹H NMR (500 MHz, CD₃OD) δ ppm 1.45 (qd, J=12.51, 4.58 Hz, 2 H), 1.76 (dd, J=12.97, 1.68 Hz, 2 H), 1.99-2.12 (m, 1 H), 3.46 (td, J=11.90, 1.83 Hz, 2 H), 3.72-3.87 (m, 5 H), 3.99 (dd, J=10.83, 3.51 Hz, 2 H), 4.52-4.82 (m, 4 H), 6.64-6.83 (m, 1 H), 6.86 (dd, J=8.09, 1.37 Hz, 2 H), 6.99 (d, J=7.02 Hz, 2 H), 7.27 (t, J=7.93 Hz, 2 H), 7.35 (br. s., 1 H), 7.42-7.60 (m, 2 H), 8.51 (br. s., 2 H); ¹³C NMR (126 MHz, CDCl₃) δ 29.75, 35.15, 55.37, 67.62, 72.57, 113.61, 113.91, 127.42, 128.82, 130.01, 137.86, 146.21, 150.16, 159.58, 161.05, 172.29. HRMS (ESI): m/z calculated for C27H₃₀N₂O₄+H⁺ [M+H⁺]: 447.2278. Found: 447.2286.

### 4-Methoxy-N-(3-phenethoxybenzyl)-N-(pyridin-4-ylmethyl)benzamide (10)

Yield=35%. ¹H NMR (500 MHz, CD₃OD) δ ppm 3.06 (t, J=6.87 Hz, 2 H), 3.82 (s, 3 H), 4.16 (br. s., 2 H), 4.51-4.80 (m, 4 H), 6.65-6.80 (m, 1 H), 6.86 (d, J=7.02 Hz, 1 H), 6.98 (d, J=7.63 Hz, 2 H), 7.21-7.38 (m, 8 H), 7.40-7.58 (m, 3 H), 8.49 (br. s., 2 H); ¹³C NMR (126 MHz, CD₃OD) δ 35.30, 54.48, 68.49, 113.66, 126.04, 127.09, 128.05, 128.48, 128.65, 129.97, 138.40, 148.81, 159.38, 161.38, 173.38. HRMS (ESI): m/z calculated for C₂₉H₂₈N₂O₃+H⁺ [M+H^{+]:} 453.2173. Found: 453.2179.

### Tert-butyl 4-((3-((4-methoxy-N-(pyridin-4-ylmethyl)benzamido)methyl)phenoxy)methyl)piperidine-1-carboxylate (11)

Yield=24%. ¹H NMR (500 MHz, CD₃OD) δ ppm 1.22-1.28 (m, 1H), 1.48 (s, 9 H), 1.79-1.81 (m, 2H), 1.93-1.95 (m, 1H), 2.77 (bs, 2H), 3.78-3.80 (m, 5H), 4.10-4.12 (m, 2H), 4.55-4.81 (m, 4 H), 6.65-6.80 (m, 1H), 6.86 (d, J=8.24 Hz, 2 H) 6.98 (d, J=7.02 Hz, 2 H) 7.22-7.38 (m, 2 H), 7.31 (bs, 1H), 7.42-7.62 (m, 2 H), 8.50 (br. s., 2 H); ¹³C NMR (126 MHz, CD₃OD) δ 28.86, 30.07, 37.55, 40.10, 56.04, 67.83, 73.48, 81.12, 115.06, 115.23, 127.72, 128.64, 128.76, 128.97, 129.43, 130.01, 130.27, 131.19, 150.43, 156.68, 161.14, 162.96, 174.96. HRMS (ESI): m/z calculated for C₃₂H₃₉N₃O₅+H⁺ [M+H⁺]: 546.2962. Found: 546.2967.

### N-(3-(cyclohexylmethoxy)benzyl)-4-methoxy-N-(pyridin-4-ylmethyl)benzamide (12)

Yield=25%. ¹H NMR (500 MHz, CD₃OD) δ ppm 1.10 (qd, J=12.16, 2.90 Hz, 2 H), 1.20-1.40 (m, 3 H), 1.69-1.83 (m, 4 H), 1.88 (d, J=13.12 Hz, 2 H), 3.71 (s, 2 H), 3.81 (s, 3 H), 4.56-4.79 (m, 4 H), 6.64-6.79 (m, 1 H), 6.85 (d, J=7.02 Hz, 2 H), 7.00 (d, J=6.41 Hz, 2 H), 7.26 (t, J=7.93 Hz, 2 H), 7.29-7.62 (m, 4 H), 8.52 (br. s., 2 H); ¹³C NMR (126 MHz, CD₃OD) δ 27.12, 27.79, 31.09, 39.24, 56.04, 74.65, 115.10, 115.23, 128.67, 130.06, 131.15, 139.27, 150.42, 161.36, 162.95, 174.96. HRMS (ESI): m/z calculated for C₂₈H₃₂N₂O₃+H⁺ [M+H⁺]: 445.2486. Found: 445.2494.

### N-(3-(4-bromophenethoxy)benzyl)-4-methoxy-N-(pyridin-4-ylmethyl)benzamide (13)

Yield=18%. ¹H NMR (500 MHz, CD₃OD) δ ppm 3.04 (t, J=6.56 Hz, 2 H), 3.83 (s, 3 H), 4.16 (br. s., 2 H), 4.53-4.78 (m, 4 H), 6.59-6.72 (m, 1 H), 6.74-6.81 (m, 1 H), 6.85-6.86 (m, 2H), 6.93-7.05 (m, 2 H), 7.24-7.27 (m, 4 H), 7.31-7.39 (bs, 1H), 7.46 (d, J=8.55 Hz, 2H), 7.49-7.55 (m, 2 H) 8.51 (br. s., 1 H); ¹³C NMR (126 MHz, CDCl₃) δ 35.17, 55.37, 68.30, 113.65, 113.91, 120.43, 127.41, 128.81, 129.01, 130.00, 130.76, 131.57, 131.79, 137.23, 137.97, 146.18, 150.19, 159.24, 161.05, 172.29. HRMS (ESI): m/z calculated for C₂₉H₂₇BrN₂O₃+H⁺ [M+H⁺]: 531.1278. Found: 531.1283.

### 4-Methoxy-N-(3-((4-nitrobenzyl)oxy)benzyl)-N-(pyridin-4-ylmethyl)benzamide (14)

Yield=35%. ¹H NMR (500 MHz, CD₃OD) δ ppm 3.82 (s, 3 H) 4.52-4.76 (m, 4 H), 5.23 (br. s., 2 H), 6.73-6.89 (m, 2 H), 6.95-6.98 (m, 3 H), 7.30 (t, J=7.93 Hz, 2 H), 7.32-7.49 (m, 3 H), 7.68 (d, J=8.85 Hz, 2 H), 8.22-8.29 (m, 2 H), 8.46-8.52 (m, 2 H); ¹³C NMR (126 MHz, CD₃OD) δ 54.47, 68.23, 113.62, 114.23, 119.74, 122.84, 123.28, 126.99, 127.53, 128.42, 129.82, 137.99, 144.99, 147.49, 148.91, 158.76, 161.39, 173.33. HRMS (ESI): m/z calculated for C₂₈H₂₅N₃O₅+H⁺ [M+H⁺]: 484.1867. Found: 484.1877.

### 4-Methoxy-N-(pyridin-4-ylmethyl)-N-(34(4-(trifluoromethyl)benzyl)oxy)benzyl)benzamide (15)

Yield=47%. ¹H NMR (500 MHz, CD₃OD) δ ppm 3.81 (s, 3 H), 4.56-4.74 (m, 4 H), 5.17 (br. s., 2 H), 6.80 (br. s., 2 H), 6.96 (d, J=7.93 Hz, 3H), 7.18-7.34 (m, 3 H), 7.45 (br. s., 2 H), 7.60-7.65 (m, 2 H), 7.66-7.72 (m, 2 H), 8.49 (br. s., 2 H); ¹³C NMR (126 MHz, CD₃OD) δ 54.46, 68.55, 113.65, 114.17, 123.18, 125.04, 125.33, 127.02, 127.28, 128.37, 129.38, 129.64, 129.77, 137.94, 141.90, 147.66, 148.88, 158.91, 161.38, 173.37. HRMS (ESI): m/z calculated for C₂₉H₂₅F₃N₂O₃+H⁺ [M+H⁺]: 507.1890. Found: 507.1898.

### N-(3-((4-fluorobenzyl)oxy)benzyl)-4-methoxy-N-(pyridin-4-ylmethyl)benzamide (16)

Yield=34%. ¹H NMR (500 MHz, CD₃OD) δ ppm 3.82 (s, 3 H), 4.51-4.77 (m, 4 H), 4.88 (s, 2 H), 6.67-6.88 (m, 2 H), 6.92-7.00 (m, 3 H), 7.08-7.14 (m, 2 H), 7.27 (t, J=8.09 Hz, 2 H), 7.33 (d, J=5.19 Hz, 1 H), 7.38-7.52 (m, 4 H), 8.49 (d, J=6.10 Hz, 2 H); ¹³C NMR (126 MHz, CD₃OD) δ 56.03, 70.35, 115.20, 115.73, 116.32, 116.50, 120.99, 124.48, 128.60, 129.95, 130.68, 130.75, 131.25, 134.81, 139.41, 149.25, 150.45, 174.92. HRMS (ESI): m/z calculated for C₂₈H₂₅FN₂O₃+H⁺ [M+H⁺]: 457.1922. Found: 457.1927.

### 4-Methoxy-N-(3-((3-methoxybenzyl)oxy)benzyl)-N-(pyridin-4-ylmethyl)benzamide (17)

Yield=27%. ¹H NMR (500 MHz, CD₃OD) δ ppm 3.79 (s, 3 H), 3.82 (s, 3 H), 4.52-4.72 (m, 4 H), 5.07 (s, 2 H), 6.76 (br. s., 1 H), 6.86-6.89 (m, 1 H), 6.92-7.01 (m, 6 H), 7.23-7.33 (m, 4 H), 7.36-7.50 (m, 2 H), 8.48 (br. s., 2 H); ¹³C NMR (126 MHz, CD₃OD) δ 113.98, 114.42, 114.88, 115.30, 115.95, 120.62, 124.42, 128.61, 129.95, 130.35, 130.82, 131.23, 139.06, 140.41, 149.16, 150.44, 160.70, 161.54, 162.90, 174.91. HRMS (ESI): m/z calculated for C₂₉H₂₈N₂O₄+H⁺ [M+H⁺]: 469.2122. Found: 469.2128.

### N-(3-(2-(4-chlorophenyl)-2-oxoethoxy)benzyl)-4-methoxy-N-(pyridin-4-ylmethyl)benzamide (18)

Yield=47%. ¹H NMR (500 MHz, CD₃OD) δ ppm 3.81 (s, 3 H), 4.51-4.76 (m, 4 H), 5.42 (br. s., 2 H), 6.66-6.88 (m, 2 H), 6.88-7.02 (m, 3 H), 7.19-7.49 (m, 4 H), 7.40-7.53 (m, 1 H), 7.54-7.60 (m, 2 H), 8.00-8.06 (m, 2 H), 8.47 (br. s., 2 H); ¹³C NMR (126 MHz, CDCl₃) δ 31.99, 55.32, 70.67, 126.69, 127.26, 128.53, 128.72, 129.23, 129.56, 132.67, 133.50, 140.51, 141.88, 146.11, 146.13, 150.12, 158.35, 161.00, 161.03, 172.25, 193.33. HRMS (ESI): m/z calculated for C₂₉H₂₅ClN₂O₄+H⁺ [M+H⁺]: 501.1576. Found: 501.1586.

### 4-Chloro-N-(3-(4-fluorophenethoxy)benzyl)-N-(pyridin-4-ylmethyl)benzamide (19)

Yield=39%. ¹H NMR (500 MHz, CD₃OD) δ ppm 3.04 (t, J=6.56 Hz, 2 H), 4.13 (br. s., 2 H), 4.50 (br. s., 2 H), 4.71 (br. s., 2H), 6.60-6.72 (m, 1H), 6.81-6.85 (m, 2H), 7.00 (t, J=8.70 Hz, 2 H), 7.18-7.32 (m, 5H), 7.41-7.49 (m, 4H), 8.46 (br. s., 2 H); ¹³C NMR (126 MHz, CDCl₃) δ 35.97, 69.94, 114.76, 115.23, 116.05, 116.22, 120.63, 124.53, 130.20, 131.32, 131.84, 131.90, 135.50, 135.97, 135.99, 137.43, 150.40, 160.91, 164.18, 173.78. HRMS (ESI): m/z calculated for C₂₈H₂₄ClFN₂O₂+H⁺ [M+H⁺]: 475.1583. Found: 475.1586.

### N-(3-(4-fluorophenethoxy)benzyl)-2-(4-methoxyphenyl)-N-(pyridin-4-ylmethyl)acetamide (20)

Yield=40%. ¹H NMR (500 MHz, CD₃OD) δ ppm 3.02 (t, J=6.71 Hz, 2 H), 3.72-3.84 (m, 5 H), 4.03 (t, J=6.71 Hz, 1 H), 4.07 (t, J=6.56 Hz, 1 H), 4.59 (s, 2 H), 4.64 (d, J=2.75 Hz, 2 H), 6.49 (s, 1 H), 6.68-6.74 (m, 1 H), 6.82 (d, J=8.54 Hz, 2 H), 6.88 (d, J=8.54 Hz, 1 H), 6.99-7.06 (m, 2 H), 7.10-7.22 (m, 5 H), 7.30 (ddd, J=8.54, 5.49, 2.75 Hz, 2 H), 8.42 (dd, J=17.70, 5.19 Hz, 2 H); ¹³C NMR (126 MHz, CDCl₃) δ 34.94, 34.97, 40.03, 40.22, 47.70, 48.60, 49.36, 50.94, 53.44, 55.31, 68.52, 68.64, 112.73, 113.72, 114.02, 114.29, 115.18, 115.23, 115.35, 115.40, 118.87, 120.71, 121.30, 122.78, 126.40, 126.58, 129.70, 129.80, 130.16, 130.40, 130.47, 133.82, 133.84, 133.96, 137.55, 138.34, 145.87, 146.34, 150.02, 150.36, 158.71, 159.14, 159.46, 160.75, 162.70, 171.92, 172.21. HRMS (ESI): m/z calculated for C₃₀H29FN₂O₃+H⁺ [M+H⁺]: 485.2235. Found: 485.2242.

### N-(3-(2-(3-bromophenyl)-2-oxoethoxy)benzyl)-4-methoxy-N-(pyridin-4-ylmethyl)benzamide (21)

Yield=36%. ¹H NMR (500 MHz, CD₃OD) δ ppm 3.81-3.86 (m, 3 H), 4.48-4.81 (m, 5 H), 5.45 (br. s., 1 H), 6.71-6.89 (m, 2 H), 6.90-7.03 (m, 3 H), 7.25-7.35 (m, 3 H), 7.41-7.55 (m, 3 H), 7.84-7.88 (m, 1 H), 8.03-8.08 (m, 1 H), 8.20 (t, J=1.68 Hz, 1 H), 8.49 (br. s., 2 H); HRMS (ESI): m/z calculated for C₂₉H₂₅BrN₂O₄+H⁺ [M+H⁺]: 545.1070. Found: 545.1072.

### 4-Methoxy-N-(3-((3-nitrobenzyl)oxy)benzyl)-N-(pyridin-4-ylmethyl)benzamide (22)

Yield=39%. ¹H NMR (500 MHz, CDCl₃) δ ppm 3.84 (s, 3 H), 4.48-4.78 (m, 4 H), 5.17 (s, 2 H), 6.81-6.97 (m, 5 H), 7.10-7.25 (m, 2 H), 7.32 (t, J=7.93 Hz, 1 H), 7.48 (br. s., 2 H), 7.61 (t, J=7.93 Hz, 1 H), 7.79 (dd, J=7.63, 0.61 Hz, 1 H), 8.22 (dd, J=8.09, 1.37 Hz, 1 H), 8.34 (s, 1 H), 8.61 (d, J=4.88 Hz, 2 H); ¹³C NMR (126 MHz, CDCl₃) δ 55.33, 68.61, 113.89, 122.11, 122.98, 127.27, 128.76, 129.62, 130.14, 133.08, 138.28, 138.91, 146.12, 148.45, 150.16, 158.65, 161.05, 172.27. HRMS (ESI): m/z calculated for C₂₈H₂₅N₃O₅+[M+H⁺]: 484.1867. Found: 484.1875.

### Compound Decrease Proliferation of MM Cells:

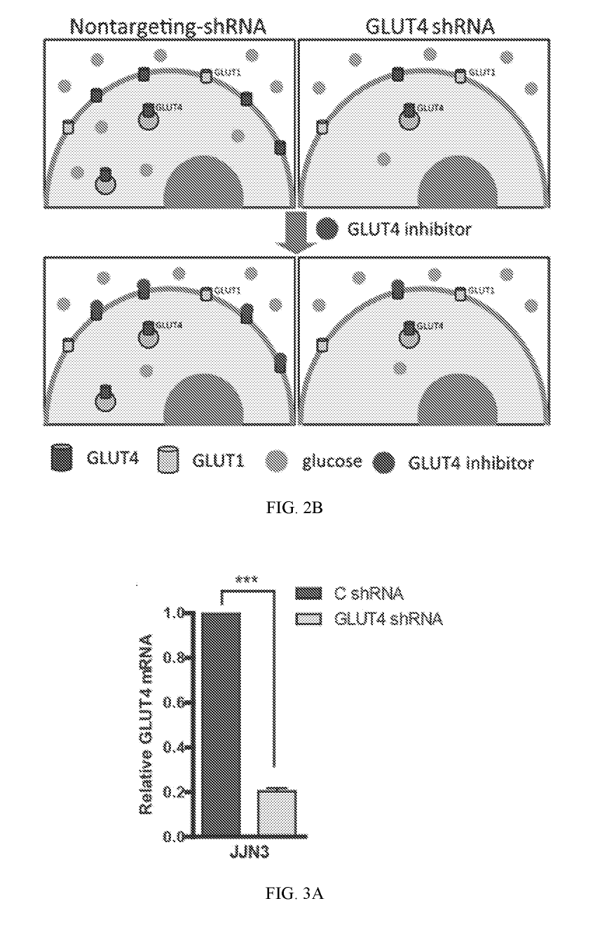

Compounds that demonstrated selectivity for GLUT4 over GLUT1 were identified. Compound were selected as a lead based on its low IC₅₀, selectivity for inhibition of glucose transport through GLUT4, and inhibition of the KMS11-GFP cell line proliferation in contrast to the GLUT1 expressing isogenic line. Compounds were assessed in a high-throughput proliferation-based assay using an isogenic MM (KMS11) cell line pair overexpressing GLUT1 or GFP (as a control) (model of screen is depicted in FIG. 2A). GLUT4 expression was determined to be maintained equally in both GFP and GLUT1 cells and the introduction of GLUT1 was found to increase proliferation and glucose uptake. Although it is not intended that certain embodiments of this disclosure be limited by any particular mechanism, it is believed that a GLUT4 targeting compound would be more effective in impacting growth of KMS11-GFP cells as opposed to KMS11-GLUT1 (as glucose transport inhibition would be compensated by glucose entry via GLUT1). Therefore, a compound with GLUT1 inhibitory activity would be more effective in KMS11-GLUT1 cells and compounds not selective for GLUT1 or 4 would likely impact viability of both cell types equally. The analogues synthesized and their IC₅₀ values for inhibition of proliferation of the isogenic lines are presented in Table 1. Isogenic KMS11-GFP/GLUT1 overexpressing myeloma cell lines were treated with a dose range of the indicated test compound for 72 hours followed by evaluation of viable cell number by Cell Titre Glo assay

TABLE 1
Evaluation of IC₅₀ for inhibition of proliferation.

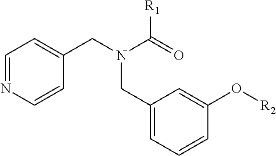

| | | | KMS11-GFP | KMS11-GLUT1 |
|---|---|---|---|---|
| Compound | R1 | R2 | EC50 (μM) | EC50 (μM) |
| 7 | 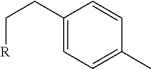 | 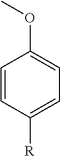 | 9.1 | 12.7 |
| 8 | 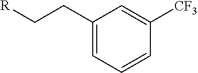 | 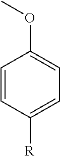 | 8.9 | 15.1 |
| 9 | 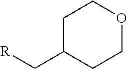 | 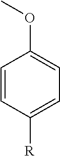 | 17.7 | 25.8 |
| 10 | 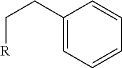 | 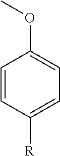 | 16.9 | 21 |
| 11 | 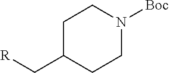 | 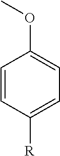 | 5.1 | 4.9 |
| 12 | 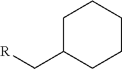 | 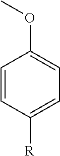 | 19.7 | 16 |
| 13 | 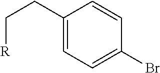 | 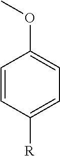 | 8.4 | 6.5 |
| 14 | 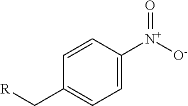 | 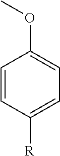 | 15.4 | 11.7 |
| 15 | 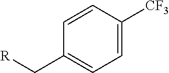 | 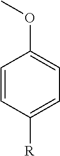 | 14.3 | 16.8 |
| 16 | 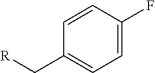 | 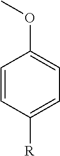 | 22.7 | 28.6 |
| 17 | 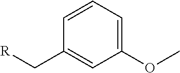 | 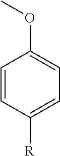 | 16.9 | 28.5 |
| 18 | 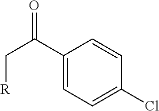 | 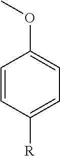 | 18.5 | 16.9 |
| 19 | 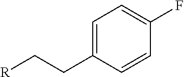 | 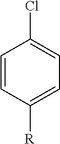 | 13.1 | 15.6 |
| 20 | 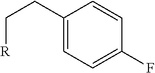 | 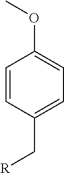 | 13.4 | 21.8 |
| 21 | 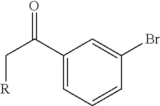 | 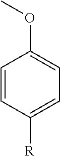 | 18.7 | 21.4 |
| 22 | 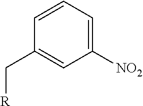 | 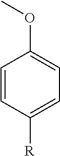 | 19.6 | 20.6 |

Several compounds (8, 9, 17, and 20) that were found to elicit a greater impact on proliferation in KMS11-GFP cells compared with KMS11-GLUT1 cells. They were selected and their effect on cell viability evaluated by AnnexinV/DAPI staining and flow cytometric analysis. Compound 20 was selected for further evaluation as it had a greater impact on the proliferation of KMS11-GFP cells (IC₅₀=13.41 μM) in contrast to the KMS11-GLUT1 cells (IC₅₀=21.83 μM) in addition to eliciting greater cell death in KMS11 cells in contrast to the other compounds.

### Compound 20 Inhibits Glucose Uptake and GLUT4-Driven Proliferation in MM:

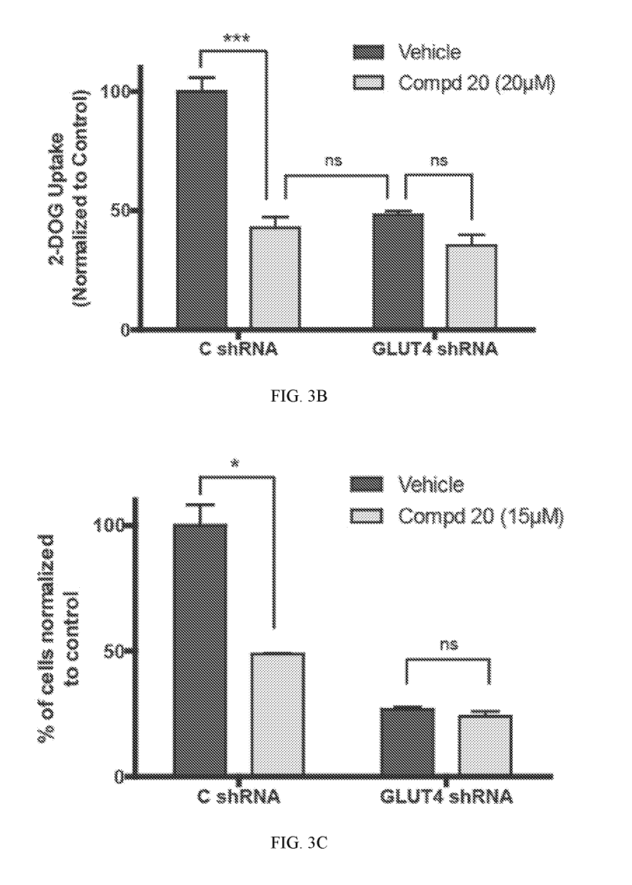

Impact of compound 20 was evaluated on glucose uptake in MM cells. Compound 20 effectively reduced [3H]2-deoxy-D-glucose (2-DOG, a glucose analog that is transported but not metabolized) uptake in JJN3 cells to a level comparable to that in JJN3 cells exhibiting GLUT4 suppression with GLUT4-specific shRNA (FIG. 3B, efficiency of knockdown shown in FIG. 3A). The impact of compound 20 was evaluated on the proliferation of cells exhibiting suppression of GLUT4 expression. Indeed, GLUT4 knockdown led to suppression of proliferation as anticipated, with no further reduction in viable cell counts upon treatment with compound 20 (FIG. 3C), underscoring the specificity of compound 20 for GLUT4.

### Compound 20 is a Selective Inhibitor of GLUT4-Mediated Glucose Transport:

Compound 20 was further screened for GLUT4 selectivity by evaluating inhibition of glucose transport in HEK 293 cells exogenously over-expressing human GLUTs-1, -2, -3, -4, or -8 that also stably express GLUT1 shRNA (except the GLUT1 overexpressing cell line) to knock down endogenous GLUT1. Preincubation of cells with a range of inhibitor, followed by a 4 minute uptake of 2-DOG indicates that compound 20 is selective for GLUT4 over GLUTs 1, 2, 3 and 8 (FIG. 4). A summary of the IC₅₀ for inhibition of glucose transport generated with analogues is presented in Table 2.

TABLE 2

| | IC₅₀ (inhibition of | | |
|---|---|---|---|
| | 2-DOG transport in HEK cells | | |
| TABLE 2: | over-expressing GLUT isoforms) | | |
| Compd I.D | GLUT1 | GLUT4 | GLUT8 |
| 3* | >100 μM | 18.9 μM | >100 μM |
| 17* | >100 μM | 10.8 μM | >100 μM |
| 26* | >100 μM | 3.5 μM | 6 μM |
| 39* | 30 μM | 30 μM | >100 μM |
| 20* | >100 μM | 18.2 μM | >100 μM |

Table 2 shows data indicating compound 20 inhibits glucose uptake by selectively targeting GLUT4. Glucose transport in HEK293 cells over-expressing GLUT1 or over-expressing GLUT4 or GLUT8 in cells lacking GLUT1 were used to evaluate inhibition of glucose transport. Cells were pre-treated with compound for 5 minutes followed by evaluation of 22-DOG uptake for 4 min at 370 C. Non-specific uptake was measured in nontransfected HEK293 cells containing the shRNA GLUT1 knockdown and was subtracted from the experimental values.

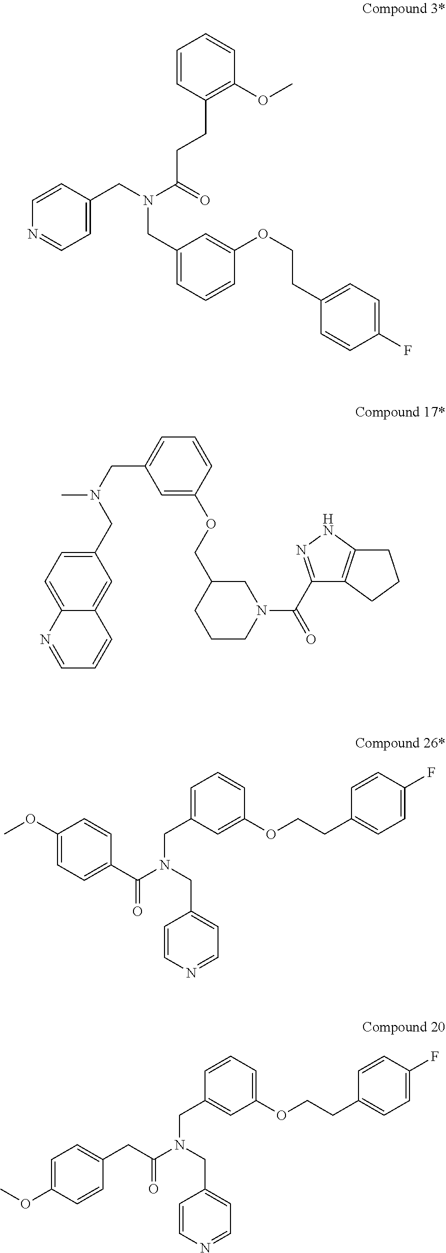 [SMILES: COc1ccccc1CCC(=O)N(Cc1ccncc1)Cc1cccc(OCCc2ccc(F)cc2)c1 ; CN(Cc1cccc(OCC2CCCN(C(=O)c3n[nH]c4c3CCC4)C2)c1)Cc1ccc2ncccc2c1 ; COc1ccc(C(=O)N(Cc2ccncc2)Cc2cccc(OCCc3ccc(F)cc3)c2)cc1 ; COc1ccc(CC(=O)N(Cc2ccncc2)Cc2cccc(OCCc3ccc(F)cc3)c2)cc1]

### Compound 20 Selectively Binds to GLUT4 vs GLUT1:

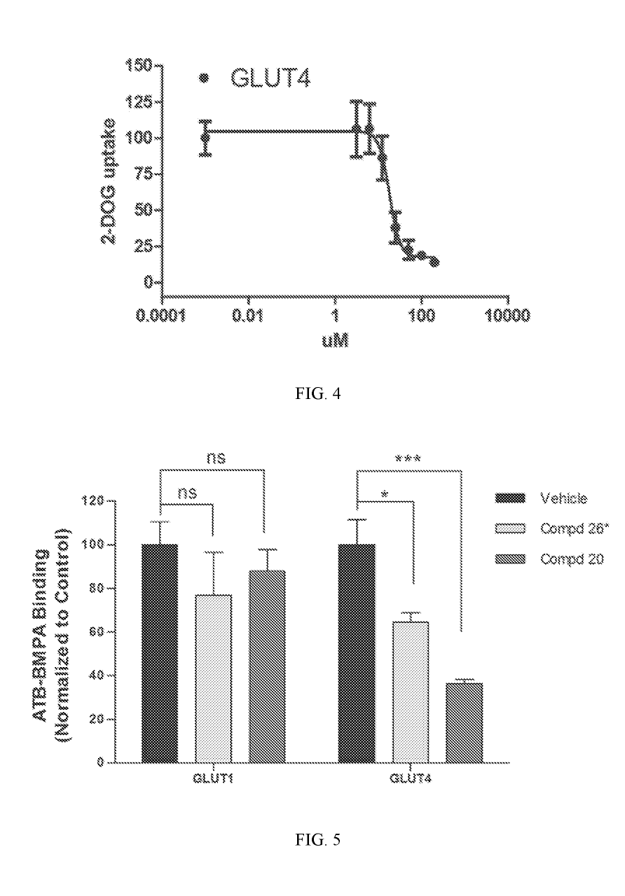

To confirm the ability of compound 20 to bind selectively to GLUT4, an established competitive binding assay that allows targeting of the glucose-binding site of GLUTs from the cytoplasmic side was utilized. Specifically, the biotinylated membrane impairment bis-mannose photolabel ATB-BMPA was used to label low-density microsomes (LDM) prepared from Myc-tagged GLUT1- or GLUT4-overexpressing cells in the presence or absence of inhibitor. The level of GLUT4 expression in myeloma cells is below the sensitivity of this assay, thus precluding direct assessment of drug binding in these cells. Compound 20 reduced ATB-BMPA binding to GLUT4 to a much greater extent than to GLUT1 (˜64% vs. ˜35%) and with greater efficacy compared to compound 26 (FIG. 5). Interestingly, 2-DOG uptake and ATB-BMPA binding results were nearly identical in terms of GLUT1 and GLUT4 inhibition by these compounds, which strongly supports their isoform selectivity and inhibitory activity for GLUT4 over GLUT1 (FIG. 5).

### GLUT4 Inhibition Chemosensitizes MM to Venetoclax and Standard MM Therapeutic Agents Melphalan and Dexamethasone:

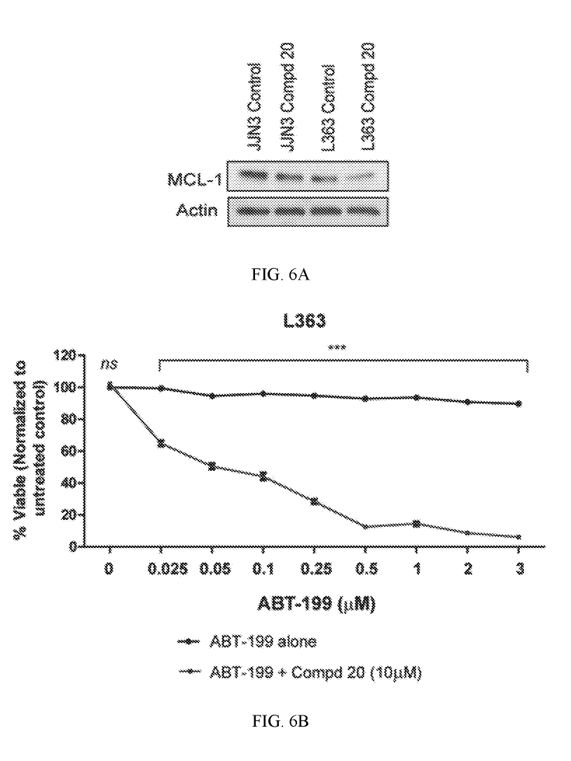

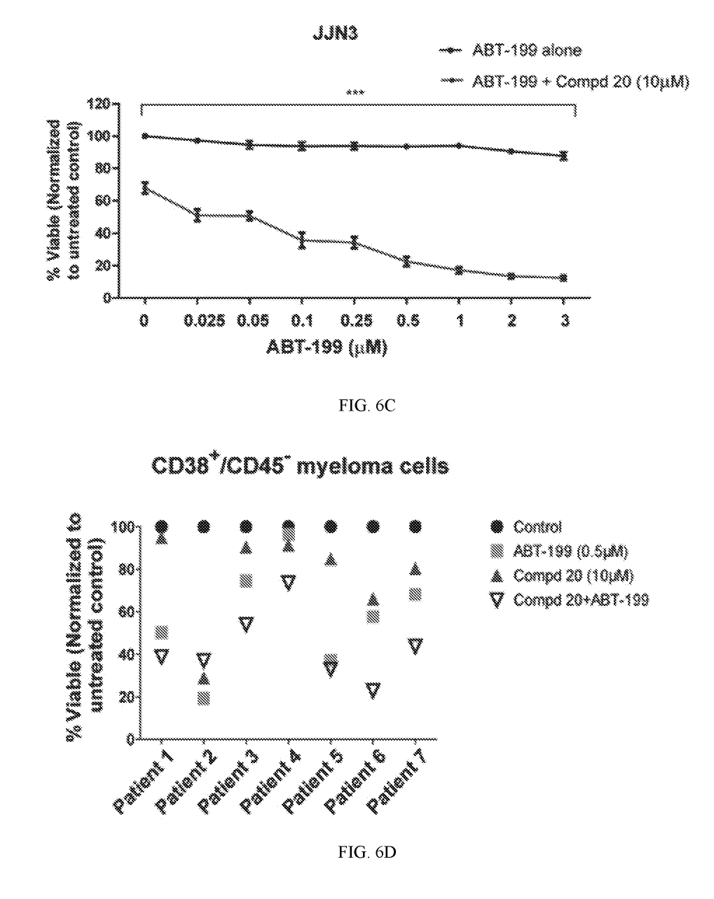

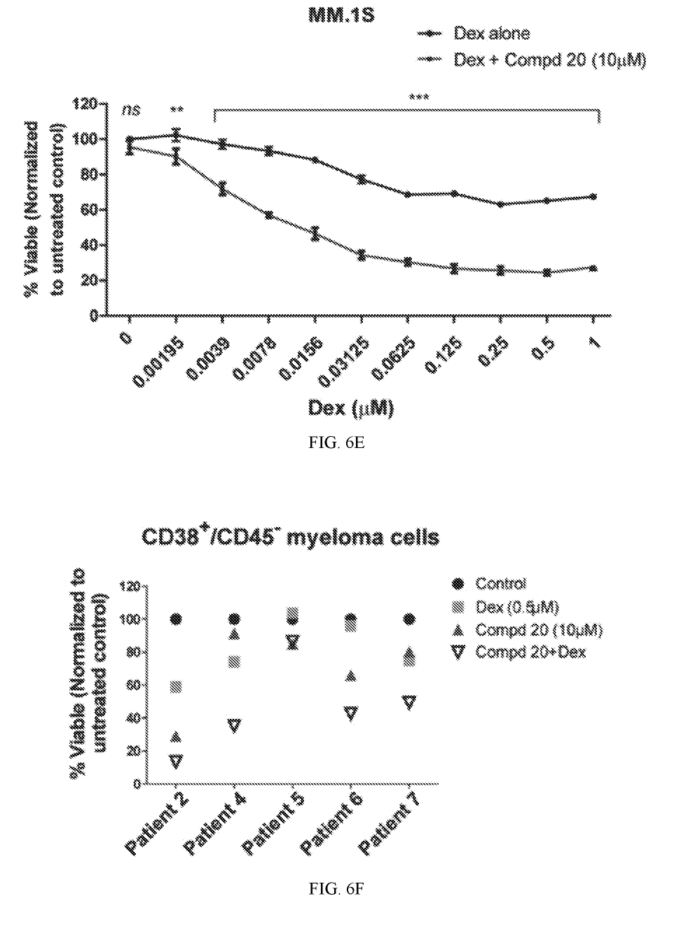

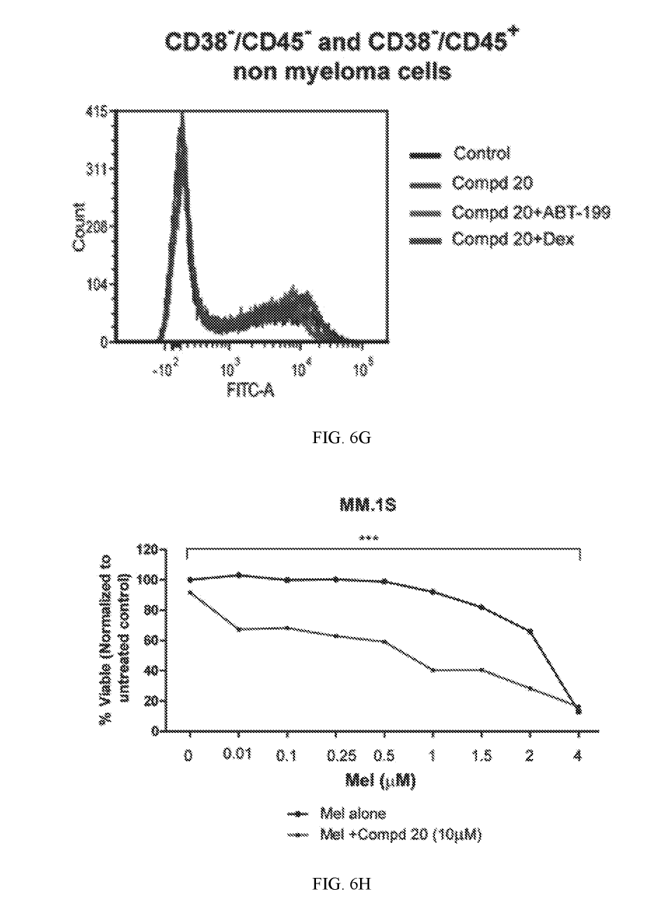

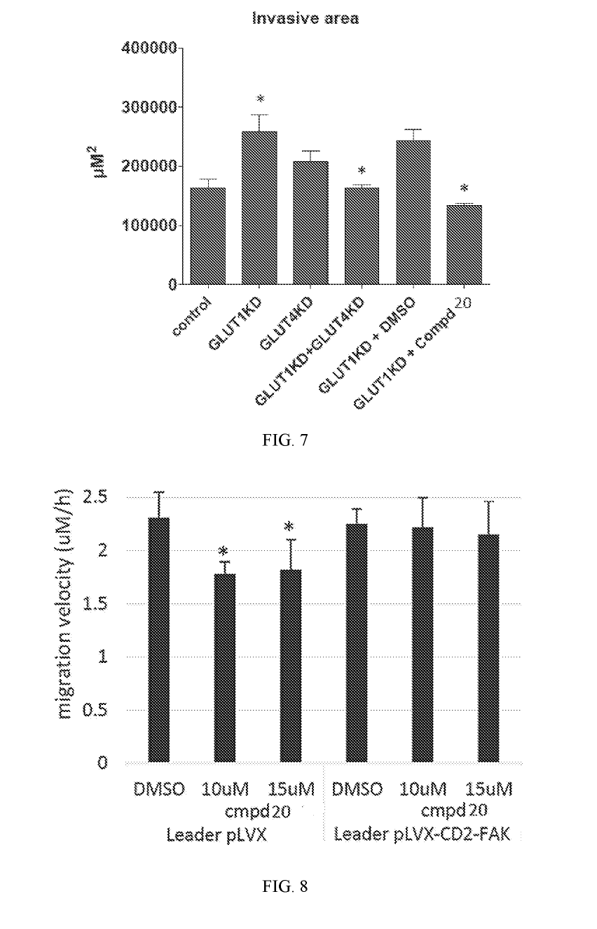

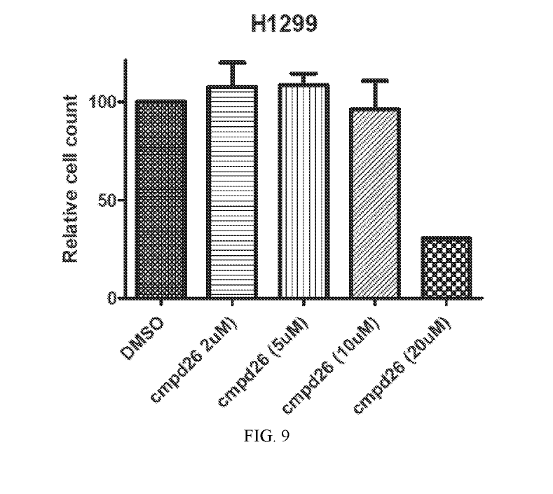

KD of GLUT4 and glucose deprivation lead to variable suppression of the pro-survival anti-apoptotic protein MCL-1. MCL-1 was significantly reduced in compound 20 treated L363 cells vs JJN3 (FIG. 6A), similar to that detected upon GLUT4 KD or glucose deprivation of these cell lines. Cells resistant to glucose deprivation-induced cell death increase binding of pro-apoptotic BIM to BCL-2, facilitating sensitization to the BH3 mimetic venetoclax (ABT-199). 22 Treatment of glucose-deprived resistant cells with ABT-199 releases the additional BIM bound to BCL-2, inducing apoptosis. Treatment with compound 20 similarly increased sensitivity to ABT-199, as demonstrated in the L363 and JJN3 cell lines (FIGS. 6B and C). Compound 20 was evaluated for sensitizing MM patient samples to ABT-199. CD38+/CD45− gated myeloma cells from myeloma patient bone marrow aspirates were evaluated for viability after 48 hrs of treatment. 4 of 7 relapse/refractory patient samples co-treated with ABT-199 and compound 20, exhibited greater apoptosis than either drug alone (FIG. 6D). The ability of compound 20 to sensitize MM cells and induce cell death to the commonly used steroid dexamethasone (Dex) was tested. Compound 20 treated MM.1S cells were exposed to a dose range of Dex (0.00195 μM to 1 μM) for 72 hr (FIG. 6E). The MM.1S cell line, which is relatively insensitive to Dex, exhibited significant sensitization and cell death to a dose range of Dex upon co-treatment with Compound 20, underscoring the significant chemosensitizing effects of GLUT4 inhibition. MM patient samples were also sensitized to the combination of 0.5 μM dexamethasone and 10 μM compound 20 (FIG. 6F). To rule out the cytotoxic effect of co-treatment of compound 20 with either ABT-199 or dexamethasone on other non-myeloma cell populations, cell viability of CD38−/CD45− and CD38−/CD45+ cells were analyzed within the MM patient sample bone marrow aspirate (FIG. 6G). Compound 20 in combination with either ABT-199 or Dex did not impact the viability of other normal non-myeloma cell populations with a representative analysis included in FIG. 6G. Lastly, compound 20 was also found to sensitize MM.15 cells to a dose range of the alkylating agent melphalan (Mel) that was assessed in a 72 hr treatment period (FIG. 6H).

What is claimed is:

1. A compound having Formula (I): 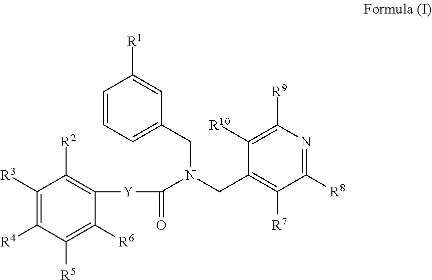
prodrugs, derivatives, or salts thereof, wherein,
Y is —CH₂—, or a direct bond from the carbonyl to the phenyl ring;
R¹ is alkoxy substituted with aryl or heterocyclyl, wherein R¹ is optionally substituted with one or more, the same or different, R¹¹;
R², R³, R⁴, R⁵, R⁶, R⁷, R⁸, R⁹, and R¹⁰ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R², R³, R⁴, R⁵, R⁶, R⁷, R⁸, R⁹, and R¹⁰ are optionally substituted with one or more, the same or different, R¹¹;
R¹¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein is optionally substituted with one or more, the same or different, R¹²; and
R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

2. The compound of claim 1 wherein R¹ is alkoxy substituted with aryl, and wherein the aryl group is optionally substituted with a halogen.

3. The compound of claim 1 which is N-(3-(4-fluorophenethoxy)benzyl)-2-(4-methoxyphenyl)-N-(pyridin-4-ylmethyl)acetamide or salts thereof.

4. The compound of claim 1 wherein R¹ is alkoxy substituted with a heterocyclyl, and wherein the heterocyclyl group is optionally substituted with R¹¹.

5. A pharmaceutical composition comprising a compound of claim 1 and a pharmaceutically acceptable excipient.

6. The pharmaceutical composition of claim 5 is in the form of a pill, tablet, capsule, or cream.

7. The pharmaceutical composition of claim 5, wherein the pharmaceutically acceptable excipient is saccharide or polysaccharide.

8. A method of treating or preventing cancer comprising administering an effective amount of a compound of claim 1 to a subject in need thereof.

9. The method of claim 8, wherein the compound is administered in combination with a second chemotherapeutic agent.

10. The method of claim 9, wherein the second chemotherapeutic agent is venetoclax, melphalan, dexamethasone, or combinations thereof.

11. The method of claim 9, wherein the second chemotherapeutic agent is gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin bortezomib anagrelide, tamoxifen, toremifene, raloxifene, droloxifene, iodoxyfene fulvestrant, bicalutamide, flutamide, nilutamide, cyproterone, goserelin, leuprorelin, buserelin, megestrol anastrozole, letrozole, vorazole, exemestane, finasteride, marimastat, trastuzumab, cetuximab, dasatinib, imatinib, bevacizumab, combretastatin, thalidomide, and/or lenalidomide or combinations thereof.

12. The method of claim 1, wherein the cancer is selected from the group consisting of multiple myeloma, leukemia, cervical, ovarian, colon, breast, gastric, skin, ovarian, pancreatic, prostate, head, neck, renal, and lung cancer.
